(19) United States
(12) Patent Application Publication
(10) Pub. No.: US 2013/0251795 A1
(43) Pub. Date: Sep. 26, 2013

(54) PHARMACEUTICAL COMPOSITIONS CONTAINING A BIGUANIDE AND A LOW DOSE ANTIDIABETIC AGENT

(71) Applicant: RANBAXY LABORATORIES LIMITED, New Delhi (IN)

(72) Inventors: Sandeep Kumar VATS, Sonipat City (IN); Balaram MONDAL, East Midnapore (IN); Kalaiselvan RAMARAJU, Tiruchirappalli (IN); Romi Barat SINGH, Varanasi (IN); Ajay Kumar SINGLA, Gurgaon (IN)

(73) Assignee: RANBAXY LABORATORIES LIMITED, New Delhi (IN)

(21) Appl. No.: 13/892,540

(22) Filed: May 13, 2013

Related U.S. Application Data
(63) Continuation-in-part of application No. 13/193,705, filed on Jul. 29, 2011.

(30) Foreign Application Priority Data
Jul. 30, 2010 (IN) 1811/DEL/2010
Mar. 25, 2011 (IN) 842/DEL/2011

(51) Int. Cl. A61K 9/00; A61K 31/155; A61K 31/4439

(52) U.S. Cl. CPC A61K 9/0002; A61K 31/4439; A61K 31/155
USPC 424/465; 424/472; 514/342; 514/635

(57) ABSTRACT

The present invention relates to pharmaceutical compositions that include a combination of a biguanide present in an extended-release form and a low dose antidiabetic agent present in an immediate-release form. The present invention further relates to processes for preparing such compositions.

### CROSS REFERENCE TO RELATED APPLICATIONS

This application is a continuation-in-part of application Ser. No. 13/193,705 claiming priority of Indian Patent Application Nos. 1811/DEL/2010 filed on Jul. 30, 2010 and 842/DEL/2011 filed on Mar. 25, 2011, all of which are incorporated herein by reference.

### FIELD OF THE INVENTION

The present invention relates to pharmaceutical compositions that include a combination of a biguanide present in an extended-release form and a low dose antidiabetic agent present in an immediate-release form. The present invention further relates to processes for preparing such compositions.

### BACKGROUND OF THE INVENTION

Two major forms of diabetes mellitus are now recognized: Type I and Type II. Type I diabetes, or insulin-dependent diabetes, is the result of an absolute deficiency of insulin, the hormone that regulates glucose utilization; patients with Type I diabetes are dependent on exogenous insulin for survival. Type II diabetes, or non-insulin-dependent diabetes (NIDDM), often occurs concurrent with normal, or even elevated levels of insulin, and appears to be the result of the inability of tissues to respond appropriately to insulin (i.e., insulin resistance). Insulin resistance is a major susceptibility trait of NIDDM and also is a contributing factor in arteriosclerosis, hypertension, lipid disorders, and polycystic ovarian syndrome.

Biguanides have been the most widely used class of antidiabetics. They act by increasing insulin activity in peripheral tissues, reducing hepatic glucose output due to inhibition of gluconeogenesis, and reducing the absorption of glucose from the intestine. Metformin, phenformin, buformin, etc. belong to this group. Metformin has been widely prescribed for lowering blood glucose in patients with NIDDM and is marketed in 500, 750, 850, and 1000 mg strengths. However, because it is a short acting drug, metformin requires twice-daily or three-times-daily dosing (500-850 mg tab 2-3 times per day or 1000 mg tab twice per day with meals). Adverse events associated with metformin include anorexia, nausea, vomiting and diarrhea. The adverse events may be partially avoided by taking an extended-release dosage form rather than multiple daily doses. Besides reducing the adverse events, administering an extended-release dosage form provides a reduction in the frequency of administration. However, to formulate a high dose biguanide like metformin HCl 1000 mg in an extended-release dosage form poses a challenge due to the large size of the dosage form. Further, the use of metformin therapy is limited by the decline in the duration of its efficacy. This problem can be solved by using a biguanide in combination with other antidiabetic agents.

Since the various antidiabetic agents act by different mechanisms, a combination therapy of a biguanide and an additional antidiabetic agent would have greater efficacy (an additive and/or synergistic effect) and as well as the possibility of reducing the adverse events as a result of using lower doses.

There are many low dose antidiabetic agents known which can be used in combination with a biguanide in an immediate-release form and enhance its efficacy and reduce the adverse events. The low dose antidiabetic agents include but are not limited to thiazolidinediones such as troglitazone, rosiglitazone, and pioglitazone; dipeptidyl peptidase-IV (DPP-IV) inhibitors such as sitagliptin, linagliptin, vildagliptin, saxagliptin, alogliptin, and dutogliptin; meglitinides such as mitiglinide, repaglinide, and nateglinide; second generation sulphonylureas such as glibenclamide, glipizide, gliclazide, and glimiperide; glucagon-like peptide (GLP-1) analogues such as exenatide; and other hypoglycaemics which are used as adjuncts to metformin therapy. However, the use of a low dose antidiabetic agent in combination with a biguanide poses a content uniformity challenge in the final dosage form.

The formulations comprising the combination of a biguanide and a low dose antidiabetic agent are commercially available under the brand names Janumet® (Sitagliptin+Metformin), Jentadueto® (Linagliptin+Metformin), Kombiglyze® XR (Saxagliptin+Metformin), Eucreas® (Vildagliptin+Metformin), Avandamet® (Rosiglitazone+Metformin), Glucovance® (Glyburide+Metformin), Metaglip® (Glipizide+Metformin) and Prandimet® (Repaglinide+Metformin).

U.S. Pat. Nos. 6,890,898, 6,803,357, 7,078,381, 7,157,429, 7,459,428, and 7,829,530 relate to the use of a combination of DPP-IV inhibitors and metformin.

U.S. Publication No. 2009/0105265 and PCT Publication No. WO 2010/092163 cover compositions comprising combinations of DPP-IV inhibitors with metformin.

U.S. Pat. No. 8,178,541, U.S. Publication No. 2011/0065731, and PCT Publication No. WO 2011/039367 cover pharmaceutical combinations and compositions comprising linagliptin and metformin.

U.S. Publication Nos. 2008/0076811 and 2011/0045062 and PCT Publication No. WO 2007/149797 cover combinations and compositions comprising vildagliptin and metformin.

U.S. Publication No. 2010/0074950 relates to a pharmaceutical composition for treating diabetes comprising a slow-release osmotic core comprising metformin and an immediate-release coating comprising a DPP-IV inhibitor.

U.S. Publication No. 2009/0221652 covers a combination of metformin and meglitinide.

U.S. Pat. No. 6,677,358 relates to combinations of a long-acting and a short-acting hypoglycemic agent. It covers a pharmaceutical composition comprising repaglinide and metformin together with a suitable carrier.

PCT Publication No. WO 2004/069229 discloses a multilayered formulation comprising extended-release biguanide and immediate-release thiazolidinedione.

PCT Publication Nos. WO 2004/026241 and WO 99/47125, U.S. Publication Nos. 2005/0249809 and 2004/0161462, and U.S. Pat. No. 6,099,862 disclose a metformin extended-release tablet coated with an immediate-release coating containing an antihyperglycemic or a hypoglycemic drug. The metformin cores are present as osmotic cores.

Although the prior art teaches pharmaceutical compositions that contain both a biguanide and a low dose antidiabetic agent, there is a need in the art to develop drug formulations in which biguanide is present in an extended-release form and a low dose antidiabetic agent is present in an immediate-release form that involves simple methods of production and are cost effective. There is also a need for a solution to the challenges of large-sized dosage forms and content uniformity issues in the final dosage. The present invention teaches a pharmaceutical composition comprising a biguanide and a low dose antidiabetic agent that overcomes the problems in the prior art by eliminating content uniformity issues in the dosage form, and reducing the size of the drug product for increased patient compliance.

### SUMMARY OF THE INVENTION

In one general aspect, the present invention provides for a pharmaceutical composition that includes a biguanide in an extended-release form and a low dose antidiabetic agent in an immediate-release form.

In another general aspect, the present invention provides for a pharmaceutical composition of a biguanide and a low dose antidiabetic agent that includes:
(i) a biguanide core which includes a therapeutically effective amount of biguanide or its pharmaceutically effective salts and one or more pharmaceutically acceptable excipients;
(ii) optionally a seal coat;
(iii) an extended-release coat which includes one or more rate-controlling materials wherein there is no passageway in the coat;
(iv) optionally a second seal coat;
(v) a low dose antidiabetic agent coat which includes a therapeutically effective amount of a low dose antidiabetic agent or its pharmaceutically effective salts and one or more pharmaceutically acceptable excipients; and
(vi) optionally a film coat.

Embodiments of the composition may include one or more of the following features. For example, the composition may be in the form of tablets or capsules. The capsules may include one or more of aggregated particles, pellets, mini tablets, tablets, beads, or granules.

In one embodiment, the biguanide may be layered onto a pharmaceutically inert core or seed. The inert core or seed may be hydrosoluble or hydroinsoluble.

The biguanide core of the present invention may include one or more pharmaceutically acceptable excipients selected from one or more of fillers, binders, disintegrants, anti adherents, lubricants, glidants, osmogents, coloring agents, and flavoring agents.

The biguanide core may additionally contain one or more absorption enhancers and/or one or more swellable polymers.

The seal coat may be applied over the biguanide core or over the extended-release coat.

The rate-controlling materials used in the composition include hydrophilic polymers, hydrophobic polymers, water-swellable polymers, other hydrophobic materials, or mixtures thereof.

The biguanide used in the composition may include metformin, phenformin, or buformin, and their pharmaceutically acceptable salts, solvates, polymorphs, enantiomers, isomers, or mixtures thereof. For example, the biguanide is metformin or its pharmaceutically acceptable salts, solvates, polymorphs, enantiomers, isomers, or mixtures thereof.

The low dose antidiabetic agent used in the composition may include DPP-IV inhibitors such as sitagliptin, linagliptin, vildagliptin, saxagliptin, alogliptin, or dutogliptin; meglitinides such as mitiglinide, repaglinide, or nateglinide; second generation sulphonylureas such as glibenclamide, glipizide, gliclazide, or glimiperide; glucagon-like peptide (GLP-1) analogues such as exenatide; other hypoglycaemics which are used as an adjunct to metformin therapy; or mixtures thereof. Particularly, the low dose antidiabetic agent is a DPP-IV inhibitor or its pharmaceutically acceptable salts, solvates, polymorphs, enantiomers, isomers, or mixtures thereof. More particularly, the low dose antidiabetic agent is linagliptin.

The biguanide core may include a mixture of biguanide and a low dose antidiabetic agent or the low dose antidiabetic agent coat may include a mixture of a low dose antidiabetic agent and biguanide or both the biguanide core and a low dose antidiabetic agent coat may include a mixture of biguanide and a low dose antidiabetic agent.

The pharmaceutical composition of the present invention may further include an additional antidiabetic agent.

In another aspect, the present invention relates to a pharmaceutical composition of a biguanide and a low dose antidiabetic agent that includes:
(i) a biguanide core which includes a therapeutically effective amount of a biguanide or its pharmaceutically effective salts and one or more pharmaceutically acceptable excipients;
(ii) optionally a seal coat;
(iii) an extended-release coat which includes one or more rate-controlling materials wherein there is no passageway in the coat;
(iv) optionally a second seal coat;
(v) an immediate-release drug coat which includes a biguanide and a low dose antidiabetic agent or its pharmaceutically effective salts and one or more pharmaceutically acceptable excipients; and
(vi) optionally a film coat.

In one embodiment, the ratio of biguanide in the core to that in the coat ranges from about 99:1 to about 60:40.

In another general aspect, the present invention provides for a process for preparing a pharmaceutical composition. The process includes the steps of:
(i) preparing the cores which include a biguanide or its pharmaceutically effective salts and one or more pharmaceutically acceptable excipients;
(ii) optionally coating the cores with a seal coat;
(iii) coating the biguanide cores of step (ii) with an extended-release coating composition which includes one or more rate-controlling materials;
(iv) coating the extended-release core with an immediate-release coating composition which includes a low dose antidiabetic agent or its pharmaceutically effective salts and one or more pharmaceutically acceptable excipients; and
(v) optionally providing a film coat.

In another general aspect, the present invention provides for a method for treating diabetes by administering to a person in need thereof a pharmaceutical composition of a biguanide and a low dose antidiabetic agent. The composition includes:
(i) a biguanide core which includes a therapeutically effective amount of biguanide or its pharmaceutically effective salts and one or more pharmaceutically acceptable excipients;
(ii) optionally a seal coat;
(iii) an extended-release coat which includes one or more rate-controlling materials wherein there is no passageway in the coat;
(iv) a low dose antidiabetic agent coat which includes a therapeutically effective amount of a low dose antidiabetic agent or its pharmaceutically effective salts and one or more pharmaceutically acceptable excipients; and
(v) optionally a film coat.

The details of one or more embodiments of the inventions are set forth in the description below. Other features and objects of the invention will be apparent from the description and examples.

### DESCRIPTION OF THE INVENTION

The present invention provides for a pharmaceutical composition that includes a biguanide in extended-release form and a low dose antidiabetic agent in an immediate-release form.

The present invention also provides for a pharmaceutical composition of a biguanide and a low dose antidiabetic agent that includes:
(i) a biguanide core which includes a therapeutically effective amount of biguanide or its pharmaceutically effective salts and one or more pharmaceutically acceptable excipients;
(ii) optionally a seal coat;
(iii) an extended-release coat which includes one or more rate-controlling materials wherein there is no passageway in the coat;
(iv) optionally a second seal coat;
(v) a low dose antidiabetic agent coat which includes a therapeutically effective amount of a low dose antidiabetic agent or its pharmaceutically effective salts and one or more pharmaceutically acceptable excipients; and
(vi) optionally a film coat.

The biguanide used in the composition may include metformin, phenformin, or buformin and their pharmaceutically acceptable salts, solvates, polymorphs, enantiomers, isomers, or mixtures thereof. For example, the biguanide may be metformin or its pharmaceutically acceptable salts, solvates, polymorphs, enantiomers, isomers, or mixtures thereof. The amount of the biguanide in the present composition may range from about 25 mg to about 2,000 mg.

The extended-release biguanide core may be present as aggregated particles, pellets, mini tablets, tablets, beads, or granules. Alternatively, the biguanide may be layered onto a pharmaceutically inert core or seed. The inert core or seed may be hydrosoluble or hydroinsoluble.

The hydrosoluble inert cores may include soluble cores, such as sugar spheres having sugars like dextrose, lactose, anhydrous lactose, spray-dried lactose, lactose monohydrate, mannitol, starches, sorbitol, sucrose, or mixtures thereof.

The hydroinsoluble cores may include one or more of glass particles/beads or silicon dioxide, calcium phosphate dihydrate, dicalcium phosphate, calcium sulfate dihydrate, microcrystalline cellulose (e.g., Avicel®), silicified microcrystalline cellulose (e.g., ProSolv®), cellulose derivatives, powdered cellulose, or mixtures thereof.

The biguanide core of the present invention includes one or more pharmaceutically acceptable excipients. The pharmaceutically acceptable excipients are known to those skilled in the art and may be selected from one or more of fillers, diluents, binders, disintegrants, anti adherents, lubricants, glidants, osmogents, coloring agents, and flavoring agents.

Examples of fillers or diluents include calcium phosphate dihydrate, calcium sulfate dihydrate, microcrystalline cellulose, cellulose derivatives, dextrose, lactose, anhydrous lactose, spray-dried lactose, lactose monohydrate, mannitol, starches, sorbitol, and sucrose. Further examples of diluents include sodium chloride, hydroxypropylmethylcellulose, hydroxypropylcellulose, methyl cellulose, carboxymethyl cellulose, sodium carboxymethyl cellulose, polyvinylpyrrolidone, polyethylene glycol, cellulose acetate butyrate, hydroxyethyl cellulose, ethyl cellulose, polyvinyl alcohol, polypropylene, dextrans, dextrins, hydroxypropyl-beta-cyclodextrin, chitosan, copolymers of lactic and glycolic acid, lactic acid polymers, glycolic acid polymers, polyorthoesters, polyanyhydrides, polyvinyl chloride, polyvinyl acetate, ethylene vinyl acetate, lectins, carbopols, silicon elastomers, polyacrylic polymers, maltodextrins, fructose, inositol, trehalose, maltose raffinose, and alpha-, beta-, and gamma-cyclodextrins, or mixtures thereof.

Examples of binders include povidones, starches, corn starch, pregelatinized starch, microcrystalline celluloses (MCC), silicified MCC (e.g., ProSolv HD® 90), microfine celluloses, lactose, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, stearic acid, gums, hydroxypropyl methylcelluloses or hypromelloses (e.g., Klucel® EF or Methocel® E5 Premium), or mixtures thereof.

Examples of disintegrants include starch, croscarmellose sodium, crospovidone, sodium starch glycolate, or mixtures thereof.

Examples of anti adherents include magnesium stearate, talc, calcium stearate, glyceryl behenate, polyethylene glycols, hydrogenated vegetable oil, mineral oil, stearic acid, and combinations thereof.

Examples of lubricants and glidants include colloidal anhydrous silica, stearic acid, magnesium stearate, calcium stearate, talc, hydrogenated castor oil, sucrose esters of fatty acids, microcrystalline wax, yellow beeswax, white beeswax, sodium benzoate, sodium acetate, sodium chloride, or mixtures thereof.

Examples of osmogents include sodium or potassium chloride; sodium or potassium hydrogen phosphate; sodium or potassium dihydrogen phosphate; salts of organic acids such as sodium or potassium acetate, magnesium succinate, sodium benzoate, sodium citrate or sodium ascorbate; carbohydrates such as mannitol, sorbitol, arabinose, ribose, xylose, glucose, fructose, mannose, galactose, sucrose, maltose, lactose, or raffinose; water-soluble amino acids such as glycine, leucine, alanine, or methionine; urea and the like; a polymer consisting of acrylic acid lightly cross-linked with polyallylsucrose; or mixtures thereof. The active ingredient, for example a biguanide, may itself act as an osmogent and facilitate the drug release.

The coloring agents and flavoring agents of the present invention may be selected from any FDA approved colors and flavors for oral use.

The biguanide core may additionally contain one or more absorption enhancers. The absorption enhancers include surfactants (anionic, cationic, or amphoteric), bile salts, calcium chelating agents, fatty acids, cyclodextrins, chitosan, or mixtures thereof. Particularly, the absorption enhancers include surfactants.

The biguanide core may additionally contain one or more swellable polymers. The term “swellable polymer” refers to polymers that gel, swell, or expand in the presence of water or biological fluids. The swellable polymers include high molecular weight hydroxpropyl methylcellulose, high molecular weight polyethylene oxides (such as POLYOX™ WSR-301, WSR-303 or WSR Coagulant), hydroxypropylcellulose, hydroxyethylcellulose, sodium carboxymethylcellulose, xanthan gum, polyvinyl acetate, or mixtures thereof.

The biguanide core may be prepared by any pharmaceutically acceptable technique that achieves uniform blending, e.g., dry blending, dry granulation, wet granulation, compaction, or fluidized bed granulation. For example, the core formulation of the present invention is fabricated by compression into tablets.

Examples of solvents used for preparing the biguanide core include methylene chloride, isopropyl alcohol, acetone, methanol, ethanol, water, or mixtures thereof. Preferably, purified water is used as the solvent.

The biguanide core is coated with an extended-release coating composition with percentage weight gain of about 1% to about 40% w/w.

The rate-controlling materials used in the extended-release coat composition are selected from one or more of hydrophilic polymers, hydrophobic polymers, water swellable polymers, other hydrophobic materials, or mixtures thereof. The rate-controlling material may be present in a concentration from about 1% to about 30% w/w of the total composition.

The extended-release coat is permeable to the passage of water, biological fluids, and active ingredient in the core. Further, the extended-release coat does not contain any passageway.

Examples of hydrophilic polymers include cellulose derivatives, such as hydroxypropyl methylcellulose, hydroxypropyl cellulose, hydroxyethyl cellulose, hydroxymethyl cellulose, methylcellulose, carboxymethyl cellulose, sodium carboxymethyl cellulose, carboxymethyl cellulose calcium, or combinations thereof; ammonium alginate; sodium alginate; potassium alginate; calcium alginate; propylene glycol alginate; alginic acid; polyvinyl alcohol; povidone; carbomer; xanthan gum; guar gum; locust bean gum; potassium pectate; potassium pectinate; polyvinylpyrrolidone; polysaccharide; polyalkylene oxides; polyethyleneglycols such as PEG 400, PEG 3350, PEG 6000, or mixtures thereof, more particularly PEG 400, PEG 3350, or mixtures thereof; starch and derivatives; or mixtures thereof.

Examples of hydrophobic polymers include ethyl cellulose, hydroxyethylcellulose, cellulose acetate, cellulose acetate butyrate, cellulose acetate phthalate, cellulose acetate trimellitate, hydroxypropyl methylcellulose phthalate, poly (alkyl) methacrylate, and copolymers of acrylic or methacrylic acid esters, ammonio methyacrylate copolymer (e.g., Eudragit® RL or Eudragit® RS), methyacrylic acid copolymers (e.g., Eudragit® L or Eudragit® S), methacrylic acid-acrylic acid ethyl ester copolymer (e.g., Eudragit® L 100-5), methacrylic acid esters neutral copolymer (e.g., Eudragit® NE 30 D), dimethylaminoethyl-methacrylate-methacrylic acid esters copolymer (e.g., Eudragit® E 100), vinyl methyl ether/maleic anhydride copolymers (e.g., Gantrez®), their salts and esters, polyvinyl acetate, or mixtures thereof.

Examples of water-swellable polymers include polyethylene oxide having a molecular weight of from 100,000 to 8,000,000; poly(hydroxy alkyl methacrylate) having a molecular weight of from 30,000 to 5,000,000; poly(vinyl) alcohol having a low acetal residue, which is cross-linked with glyoxal, formaldehyde, or glutaraldehyde and having a degree of polymerization of from 200 to 30,000; a mixture of methyl cellulose, cross-linked agar, and carboxymethyl cellulose; a water insoluble, water-swellable copolymer produced by forming a dispersion of a finely divided copolymer of maleic anhydride with styrene, ethylene, propylene, butylene, or isobutylene cross-linked with from 0.001 to 0.5 moles of saturated cross-linking agent per mole of maleic anhydride in the copolymer; Carbopol® carbomer which is an acidic carboxy polymer having a molecular weight of from 450,000 to 4,000,000; Cyanamer® polyacrylamides; cross-linked water-swellable indene-maleic anhydride polymers; polyacrylic acid having a molecular weight of from 80,000 to 200,000; starch graft copolymers; acrylate polymer polysaccharides composed of condensed glucose units such as diester cross-linked polyglucan and the like; Amberlite® ion exchange resins; Explotab® sodium starch glycolate; Ac-Di-Sol® croscarmellose sodium; and combinations thereof.

Examples of other hydrophobic materials include waxes such as beeswax, carnauba wax, microcrystalline wax, candelilla wax, spermaceti, montan wax, hydrogenated vegetable oil, lecithin, hydrogenated cottonseed oil, hydrogenated tallow, paraffin wax, shellac wax, petrolatum, ozokerite, and the like as well as synthetic waxes, e.g., polyethylene and the like; fatty acids such as stearic acid, palmitic acid, lauric acid, eleostearic acids, and the like; fatty alcohols such as lauryl alcohol, cetostearyl alcohol, stearyl alcohol, cetyl alcohol and myristyl alcohol; fatty acid esters such as glyceryl monostearate, glycerol monooleate, acetylated monoglycerides, tristearin, tripalmitin, cetyl esters wax, glyceryl palmitostearate and glyceryl behenate; vegetable oil such as hydrogenated castor oil; mineral oil; and combinations thereof.

The extended-release coat composition may additionally include plasticizers selected from propylene glycol, triethylene glycol, oleic acid, ethyleneglycol monoleate, triethyl citrate, triacetin, diethyl phthalate, glyceryl monostearate, dibutyl sebacate, acetyl triethyl citrate, acetyl tributyl citrate, castor oil, or mixtures thereof. It may also include opacifiers selected from titanium dioxide, talc, calcium carbonate, behenic acid, cetyl alcohol, or mixtures thereof.

The extended-release coat composition may optionally include pore formers selected from polymers like hydroxyalkyl cellulose, hydroxypropyl methylcellulose, hydroxypropyl cellulose, polyethylene glycols, polyvinyl alcohol, povidone, copovidone, poloxamers such as 188 or 407, sugars, salts, or mixtures thereof.

The coating compositions may be coated on the biguanide core by conventional methods such as drug layering, dry compression, deposition, and printing.

The coating composition may be applied as a solution or dispersion of rate-controlling materials. Examples of solvents used for preparing a solution or dispersion of rate-controlling materials include methylene chloride, isopropyl alcohol, acetone, methanol, ethanol, water, or mixtures thereof.

A seal coating composition may be coated over the biguanide core or over the extended-release coat. The polymers used to provide seal coating may include one or more hydrophilic polymers. Examples of hydrophilic polymers include hydroxypropylcellulose, hydroxypropylisopropylcellulose, methoxypropyl cellulose, hydroxypropylmethylcellulose, hydroxypropylpentylcellulose, hydroxypropylhexylcellulose, or mixtures thereof. The seal coating may optionally be opacified.

The extended-release biguanide core is coated with an immediate-release drug layer. The immediate-release drug layer comprises a therapeutically effective amount of a low dose antidiabetic agent and optionally a biguanide.

The low dose antidiabetic agents used in the composition include, but are not limited to, thiazolidinediones such as troglitazone, rosiglitazone, or pioglitazone; dipeptidyl peptidase-IV (DPP-IV) inhibitors such as sitagliptin, linagliptin, vildagliptin, saxagliptin, alogliptin, or dutogliptin; meglitinides such as mitiglinide, repaglinide, or nateglinide; second generation sulphonylureas such as glibenclamide, glipizide, gliclazide, or glimepiride glucagon-like peptide (GLP-1) analogues such as exenatide; and other hypoglycaemics which are used as an adjunct to metformin therapy. The amount of the low dose antidiabetic agent used in the present composition may range from about 1 mg to about 200 mg.

Further, a portion of biguanide may also be present in the immediate-release drug layer along with the low dose antidiabetic agent. The addition of a portion of biguanide in the coat results in reduction of core size and overall dosage form size, resulting in improved patient compliance.

The present invention also encompasses a composition comprising an extended-release biguanide layer and an immediate-release drug layer comprising a biguanide and a low dose antidiabetic agent. The biguanide used in the core and the coat may be similar or different. The ratio of biguanide in the core to that in the coat ranges from about 99:1 to about 60:40.

The term “about”, as used herein, when used along with the values assigned to certain measurements and parameters means a variation of up to 10% from such values, or in the case of a range of values, means up to a 10% variation from both the lower and upper limits of such ranges.

The low dose antidiabetic agent coat includes a therapeutically effective amount of a low dose antidiabetic agent or its pharmaceutically effective salts, optionally a biguanide, and one or more pharmaceutically acceptable excipients known to those skilled in the art, which may be selected from one or more of wicking agents, wetting agents, plasticizers, opacifiers and colorants.

A wicking agent is defined as any material with the ability to draw water into the porous network of a delivery device. Examples of wicking agents include silicon dioxide (e.g., Syloid® 244FP), kaolin, titanium dioxide, alumina, niacinamide, sodium lauryl sulfate, low molecular weight polyvinyl pyrrolidone, m-pyrol, bentonite, magnesium aluminum silicate, polyester, and polyethylene. Non-swellable wicking agents, such as sodium lauryl sulfate, colloidal silicon dioxide, and low molecular weight polyvinylpyrrolidone, are preferred.

Examples of wetting agents include hydrophilic surfactants, hydrophobic surfactants, or mixtures thereof. The hydrophilic surfactants may be selected from one or more of non-ionic surfactants, ionic surfactants, or mixtures thereof.

Examples of hydrophobic surfactants include one or more of alcohols; polyoxyethylene alkylethers; fatty acids; glycerol fatty acid monoesters; glycerol fatty acid diesters; acetylated glycerol fatty acid monoesters; acetylated glycerol fatty acid diesters; lower alcohol fatty acid esters; polyethylene glycol fatty acid esters; polyethylene glycol glycerol fatty acid esters; polypropylene glycol fatty acid esters; polyoxyethylene glycerides; lactic acid derivatives of monoglycerides; lactic acid derivatives of diglycerides; propylene glycol diglycerides; sorbitan fatty acid esters; polyoxyethylene sorbitan fatty acid esters; polyoxyethylene-polyoxypropylene block copolymers; polyethyleneglycols as esters or ethers; polyethoxylated castor oil; polyethoxylated hydrogenated castor oil; polyethoxylated fatty acid from castor oil; polyethoxylated fatty acid from hydrogenated castor oil; or mixtures thereof.

Examples of non-ionic surfactants include alkylglucosides; alkylmaltosides; alkylthioglucosides; lauryl macrogolglycerides; caprylocaproyl macrogolglycerides; polyoxyethylene alkyl ethers; polyoxyethylene alkylphenols; polyethylene glycol fatty acid esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction products of polyols and at least one member of the group consisting of fatty acids, glycerides, vegetable oils, hydrogenated vegetable oils, and sterols; sugar esters; sugar ethers; sucroglycerides; or mixtures thereof.

Examples of ionic surfactants include alkyl ammonium salts; bile acids and salts, analogues, and derivatives thereof; fatty acid derivatives of amino acids, oligopeptides, and polypeptides; glyceride derivatives of amino acids, oligopeptides, and polypeptides; acyl lactylates; monoacetylated tartaric acid esters of monoglycerides; monoacetylated tartaric acid esters of diglycerides; diacetylated tartaric acid esters of monoglycerides; diacetylated tartaric acid esters of diglycerides; succinylated monoglycerides; citric acid esters of monoglycerides;

citric acid esters of diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; or mixtures thereof.

Examples of plasticizers include propylene glycol, triethylene glycol, oleic acid, ethyleneglycol monoleate, triethyl citrate, triacetin, diethyl phthalate, glyceryl monostearate, dibutyl sebacate, acetyl triethyl citrate, acetyl tributyl citrate, castor oil, or mixtures thereof.

Examples of opacifiers include titanium dioxide, talc, calcium carbonate, behenic acid, cetyl alcohol, or mixtures thereof.

The immediate-release drug layer may further include one or more film-forming polymers. The film-forming polymers may be hydrophilic polymers.

The low dose antidiabetic agent coating composition may be applied as a solution or dispersion over the extended-release coat. Examples of solvents used for preparing a solution or dispersion of a low dose antidiabetic agent include methylene chloride, isopropyl alcohol, acetone, methanol, ethanol, water, or mixtures thereof.

The pharmaceutical composition may optionally be coated with one or more layers of a film coat comprising film forming agents and/or pharmaceutically acceptable excipients.

Examples of film forming agents include ethyl cellulose, hydroxypropyl methylcellulose, hydroxypropyl cellulose, methyl cellulose, carboxymethylcellulose, hydroxymethylcellulose, hydroxyethylcellulose, hydroxypropyl methyl phthalate, cellulose acetate, cellulose acetate trimelliatate, cellulose acetate phthalate waxes such as polyethylene glycol, methacrylic acid polymers such as Eudragit® RL and RS, or mixtures thereof. Alternatively, commercially available coating compositions comprising film-forming polymers marketed under various trade names, such as Opadry®, may also be used for coating.

The film forming agents may be applied as a solution or dispersion of coating ingredients. Examples of solvents used for preparing a solution or dispersion of the coating ingredients include methylene chloride, isopropyl alcohol, acetone, methanol, ethanol, water, or mixtures thereof.

The various coats of the composition may be coated using a conventional coating pan, a spray coater, a rotating perforated pan, an automated system such as a centrifugal fluidizing (CF) granulator, a fluidized bed process, or any other suitable automated coating equipment.

The various compositions described above are used for treating diabetes.

The present invention is illustrated below by reference to the following examples. However, one skilled in the art will appreciate that the specific methods and results discussed are merely illustrative of the invention, and not to be construed as limiting the invention.

### EXAMPLES

### Example 1

| | |
|---|---|
| Ingredients | Percent w/w of tablet |

Core Composition

| | |
|---|---|
| Metformin hydrochloride | 68.49 |
| Microcrystalline cellulose | 13.70 |
| Polyvinylpyrrolidone | 4.11 |
| Hydrophobic silicon dioxide | 0.43 |
| Magnesium stearate | 0.43 |

Extended Release (ER) Coating (10-15%)

| | |
|---|---|
| Ethyl cellulose | 8.20 |
| Hydroxypropyl methylcellulose | 0.43 |
| Talc | 0.43 |
| Isopropyl alcohol | q.s. |
| Purified water | q.s. |

Active Coat

| | |
|---|---|
| Pioglitazone hydrochloride | 2.05 |
| equivalent to pioglitazone | |
| Polysorbate-80 | 0.03 |
| Hydroxypropyl methylcellulose E-5 | 0.25 |
| Isopropyl alcohol | q.s. |
| Purified water | q.s. |

Film Coating

| | |
|---|---|
| Opadry ® White | 1.44 |
| Purified water | q.s. |

Procedure:

### Core Tablets:

1. Metformin hydrochloride and microcrystalline cellulose were mixed uniformly and granulated with a polyvinylpyrrolidone solution in water.
2. The granules were dried in the fluidized bed dryer and the dried granules were mixed with hydrophobic colloidal silicon dioxide and magnesium stearate.
3. The blend of step 2 was compressed into core tablets.

### ER Coating:

4. Ethyl cellulose, hydroxypropyl methylcellulose, and talc were dispersed in an isopropyl alcohol:water mixture.
5. The tablets of step 3 were coated with the dispersion of step 4.

### Active Coat:

6. The isopropyl alcohol:water mixture was taken in a container and hydroxypropyl methylcellulose E-5 was added to it, followed by Polysorbate-80 and pioglitazone hydrochloride.
7. The ER coated tablets were coated with the dispersion of step 6 in a pan coater.

### Film Coating:

8. The tablets of step 7 were coated with Opadry® coating.

### Example 2

| | |
|---|---|
| Ingredients | Percent w/w of tablet |

Core Composition

| | |
|---|---|
| Metformin hydrochloride | 64.52 |
| Sodium carboxymethyl cellulose | 3.23 |
| Microcrystalline cellulose | 6.45 |
| Hydroxypropyl methylcellulose | 11.29 |
| Magnesium stearate | 0.65 |
| Silicon dioxide | 0.65 |
| Hydroxypropyl methylcellulose E-5 | 1.61 |
| Purified Water | q.s. |

ER Coating (0-15%)

| | |
|---|---|
| Ethyl cellulose | 7.34 |
| Hydroxypropyl methylcellulose | 0.41 |
| Talc | 0.39 |
| Isopropyl alcohol | q.s. |
| Purified water | q.s. |

Coat Composition

| | |
|---|---|
| Pioglitazone hydrochloride | 1.94 |
| equivalent to pioglitazone | |
| Polysorbate-80 | 0.02 |
| Hydroxypropyl methylcellulose | 0.24 |
| Isopropyl alcohol | q.s. |
| Purified water | q.s. |

Film Coating

| | |
|---|---|
| Opadry ® White | 1.29 |
| Purified water | q.s. |

Procedure:

### Core Tablets:

1. Metformin hydrochloride, sodium carboxymethyl cellulose, microcrystalline cellulose and hydroxypropyl methylcellulose were mixed uniformly and granulated with hydroxypropyl methylcellulose E-5.
2. The granules were dried in a fluidized bed dryer and lubricated with magnesium stearate and silicon dioxide.
3. The blend of step 2 was compressed into core tablets.

### ER Coating:

4. Ethyl Cellulose, hydroxypropyl methylcellulose and talc were dispersed in an isopropyl alcohol:water mixture.
5. The tablets of step 3 were coated with the dispersion of step 4.

### Pioglitazone Coating:

6. The isopropyl alcohol:water mixture was taken in a container and hydroxypropyl methylcellulose was added to it followed by polysorbate-80 and pioglitazone hydrochloride.
7. The ER coated tablets were coated with the dispersion of step 6 in a pan coater.

### Film Coating:

8. The tablets of step 7 were coated with Opadry® coating.

### Example 3

| | |
|---|---|
| Ingredients | Percent w/w of tablet |

Core Composition

| | |
|---|---|
| Metformin hydrochloride | 68.49 |
| Polyvinylpyrrolidone | 4.79 |
| Sodium lauryl sulphate | 1.71 |
| Sodium chloride | 1.71 |
| Magnesium stearate | 0.68 |
| Purified water | q.s. |

ER Coating (5%-20%)

| | |
|---|---|
| Ethyl cellulose | 13.23 |
| Hydroxypropyl methylcellulose | 1.47 |
| Diethyl phthalate | 1.47 |
| Talc | 0.75 |
| Isopropyl alcohol | q.s. |
| Purified water | q.s. |

Pioglitazone Layer

| | |
|---|---|
| Pioglitazone hydrochloride | 2.05 |
| equivalent to pioglitazone | |
| Hydroxypropyl methylcellulose | 2.05 |
| Polysorbate 80 | 0.03 |
| Talc | 1.53 |
| Isopropyl alcohol | q.s. |
| Purified water | q.s. |

Procedure:

### Core Tablets:

1. Metformin hydrochloride, sodium lauryl sulphate, and sodium chloride were mixed uniformly and granulated with polyvinylpyrrolidone in a rapid mixer granulator.
2. The granules were dried in a fluidized bed dryer and lubricated with magnesium stearate.
3. The blend of step 2 was compressed into core tablets.

### ER Coating:

4. Ethyl Cellulose, hydroxypropyl methylcellulose, diethyl phthalate, and talc were dispersed in an isopropyl alcohol:water mixture.
5. The tablets of step 3 were coated with the dispersion of step 4.

### Pioglitazone Layer:

6. Pioglitazone hydrochloride, hydroxypropyl methylcellulose, polysorbate 80, and talc were dispersed in an isopropyl alcohol:water mixture.
7. The ER coated tablets were coated with the dispersion of step 6.

### Example 4

| | |
|---|---|
| Ingredients | Percent w/w of tablet |

Core Composition

| | |
|---|---|
| Metformin hydrochloride | 71.79 |
| Microcrystalline cellulose | 3.59 |
| Sodium lauryl sulphate | 3.59 |
| Polyvinylpyrrolidone | 5.03 |
| Magnesium stearate | 0.72 |
| Isopropyl alcohol | q.s. |

ER Coating

| | |
|---|---|
| Cellulose acetate | 8.69 |
| Triacetin | 0.57 |
| Polyethylene glycol | 0.57 |
| Hydroxypropyl methylcellulose | 2.87 |

Pioglitazone Layer

| | |
|---|---|
| Pioglitazone hydrochloride | 2.15 |
| equivalent to pioglitazone | |
| Sodium lauryl sulphate | 0.14 |
| Hydroxypropyl methylcellulose | 0.29 |
| Isopropyl alcohol | q.s. |
| Purified water | q.s. |

Procedure:

### Core Tablets:

1. Metformin hydrochloride, microcrystalline cellulose, and sodium lauryl sulphate were mixed uniformly and granulated with a polyvinylpyrrolidone solution in isopropyl alcohol.
2. The granules were dried in a fluidized bed dryer and lubricated with magnesium stearate.
3. The blend of step 2 was compressed into core tablets.

### ER Coating:

4. Cellulose acetate was dissolved in acetone followed by addition of triacetin, polyethylene glycol, water, and hydroxypropyl methylcellulose under stirring.
5. The tablets of step 3 were coated with the dispersion of step 4.

### Pioglitazone Layer:

6. Hydroxypropyl methylcellulose and sodium lauryl sulphate were dissolved in water followed by the addition of isopropyl alcohol.
7. Pioglitazone hydrochloride was dispersed into the solution of step 6.
8. The ER coated tablets were coated with the dispersion of step 7.

### Example 5

| | |
|---|---|
| | Percent w/w |
| Ingredients | of tablet |

Core Composition

| | |
|---|---|
| Metformin hydrochloride | 60.00-85.00 |
| Microcrystalline cellulose | 1.00-5.00 |
| Sodium lauryl sulphate | 0.50-3.00 |
| Polyvinylpyrrolidone | 4.00-7.50 |
| Magnesium stearate | 0.20-0.60 |
| Purified water/Isopropyl alcohol | q.s. |

Seal Coating (Optional)

| | |
|---|---|
| Hydroxypropyl methylcellulose | 1.00-3.00 |
| Isopropyl alcohol | q.s. |

ER Coating (3%-15% Weight Buildup)

| | |
|---|---|
| Cellulose acetate | 2.50-7.50 |
| Triacetin | 0.10-0.50 |
| Polyethylene glycol 400 | 0.20-0.60 |
| Polyethylene glycol 6000 or hydroxypropyl | 0.20-2.50 |
| methylcellulose | |
| Acetone | q.s. |
| Purified water | q.s. |

Pioglitazone Layer

| | |
|---|---|
| Pioglitazone hydrochloride equivalent to pioglitazone | 3.00-1.50 |
| Lactose monohydrate | 0.05-5.00 |
| Sodium lauryl sulphate (Optional) | 0.00-1.50 |
| Hydroxypropyl cellulose | 0.20-5.00 |
| Purified water/Non-aqueous solvent | q.s. |

Procedure:

### Core Tablets:

1. Metformin hydrochloride, microcrystalline cellulose, and sodium lauryl sulphate were mixed uniformly and granulated with a polyvinylpyrrolidone solution in isopropyl alcohol.
2. The granules were dried in a fluidized bed dryer and lubricated with magnesium stearate.
3. The blend of step 2 was compressed into core tablets.

### Seal Coating:

4. Hydroxypropyl methylcellulose was dissolved in isopropyl alcohol.
5. The tablets of step 3 were coated with the dispersion of step 4.

### ER Coating:

6. Cellulose acetate was dissolved in acetone followed by the addition of triacetin, polyethylene glycol, water, and hydroxypropyl methylcellulose under stirring.
7. The seal coated tablets of step 5 were coated with the dispersion of step 6.

### Pioglitazone Layer:

8. Lactose, hydroxypropyl cellulose, and sodium lauryl sulphate were dissolved in water/non-aqueous solvent.
9. Pioglitazone hydrochloride was dispersed into the solution of step 8.
10. The ER coated tablets were coated with the dispersion of step 9.

### Example 6

| | |
|---|---|
| Ingredients | Percent w/w of Tablet |

Core Composition

| | |
|---|---|
| min hydrochloride | |
| Microcrystalline cellulose | 3.80 |
| Polyvinylpyrrolidone | 7.60 |
| Sodium lauryl sulphate | 0.76 |
| Magnesium stearate | 0.76 |
| Isopropyl alcohol | q.s. |

Seal Coating (2.5% Weight Buildup; Range 2%-3%)

| | |
|---|---|
| Hydroxypropyl methylcellulose | 2.22 |
| Isopropyl alcohol (85 parts) | q.s. |
| Water (15 parts) | q.s. |

ER Coating: (2% Weight Buildup; Range 1%-3%)

| | |
|---|---|
| Cellulose acetate | 1.55 |
| Triacetin | 0.09 |
| Polyethylene glycol 400 | 0.18 |
| Acetone | q.s. |

Pioglitazone Coating

| | |
|---|---|
| Pioglitazone hydrochloride equivalent to | 2.51 |
| pioglitazone | |
| Lactose monohydrate | 2.28 |
| Hydroxypropylcellulose | 2.28 |
| Purified water | q.s. |

Procedure:

### Core Tablets:

1. Metformin hydrochloride, microcrystalline cellulose, and sodium lauryl sulphate were mixed uniformly and granulated with a polyvinylpyrrolidone solution in isopropyl alcohol.
2. The granules were dried in a fluidized bed dryer and lubricated with magnesium stearate.
3. The blend of step 2 was compressed into core tablets.

### Seal Coating:

4. Hydroxypropyl methylcellulose was dissolved in isopropyl alcohol.
5. The tablets of step 3 were coated with the s dispersion of step 4.

### ER Coating:

6. Cellulose acetate was dissolved in acetone followed by the addition of triacetin and polyethylene glycol under stirring.
7. The seal coated tablets of step 5 were coated with the dispersion of step 6.

### Pioglitazone Layer:

8. Lactose and hydroxypropylcellulose were dissolved in water.
9. Pioglitazone hydrochloride was dispersed into the solution of step 8.
10. The ER coated tablets were coated with the dispersion of step 9.

### Example 7

| | |
|---|---|
| Ingredients | Percent w/w of Tablet |

Core Composition

| | |
|---|---|
| Metformin HCl | 70.22 |
| Microcrystalline cellulose | 3.51 |
| Polyvinylpyrrolidone | 7.02 |
| Sodium lauryl sulphate | 0.70 |
| Magnesium stearate | 0.70 |
| Isopropyl alcohol | q.s. |

Seal Coating (2.5% Weight Buildup; Range 2%-3%)

| | |
|---|---|
| Hydroxypropyl methylcellulose | 2.05 |
| Isopropyl alcohol (85 parts) | q.s. |
| Purified water (15 parts) | q.s. |

ER Coating (8%-10% Weight Buildup)

| | |
|---|---|
| Cellulose acetate | 4.38 |
| Polyethylene glycol 6000 | 1.01 |
| Triacetin | 0.67 |
| Polyethylene glycol 400 | 0.67 |
| Acetone | q.s. |

Pioglitazone Coating

| | |
|---|---|
| Pioglitazone hydrochloride equivalent to | 2.32 |
| pioglitazone | |
| Lactose monohydrate | 2.87 |
| Hydroxypropylcellulose | 1.76 |
| Silicon dioxide | 2.11 |
| Purified water | q.s. |

Procedure:

### Core Tablets:

1. Metformin hydrochloride, microcrystalline cellulose, and sodium lauryl sulphate were mixed uniformly and granulated with a polyvinylpyrrolidone solution in isopropyl alcohol.
2. The granules were dried in a fluidized bed dryer and lubricated with magnesium stearate.
3. The blend of step 2 was compressed into core tablets.

### Seal Coating:

4. Hydroxypropyl methylcellulose was dissolved in isopropyl alcohol.
5. The tablets of step 3 were coated with the dispersion of step 4.

### ER Coating:

6. Cellulose acetate was dissolved in acetone followed by the addition of triacetin and polyethylene glycol under stirring.
7. The seal coated tablets of step 5 were coated with the dispersion of step 6.

### Pioglitazone Layer:

8. Lactose, hydroxypropylcellulose, and silicon dioxide were dissolved in water.
9. Pioglitazone hydrochloride was dispersed into the solution of step 8.
10. The ER coated tablets were coated with the dispersion of step 9.

### Example 8

| | | |
|---|---|---|
| | Percent | Percent |
| | w/w of Tablet | w/w of Tablet |
| Ingredients | (15/1000 mg) | (30/1000 mg) |

Core Composition

| | | |
|---|---|---|
| Metformin hydrochloride | 72.60 | 71.73 |
| Sodium lauryl sulphate | 2.90 | 2.87 |
| Polyvinylpyrrolidone | 7.26 | 7.17 |
| Purified water | q.s. | q.s. |
| Magnesium stearate | 0.41 | 0.41 |

Seal Coating

| | | |
|---|---|---|
| Hydroxypropyl | 1.66 | 1.64 |
| methylcellulose | | |
| Isopropyl alcohol | q.s. | q.s. |
| Purified water | q.s. | q.s. |

ER coating

| | | |
|---|---|---|
| Cellulose acetate | 4.96 | 4.90 |
| Polyethylene glycol 6000 | 1.15 | 1.13 |
| Triacetin | 0.76 | 0.75 |
| Polyethylene glycol 400 | 0.76 | 0.75 |
| Acetone | q.s. | q.s. |
| Purified water | q.s. | q.s. |

Pioglitazone Coating

| | | |
|---|---|---|
| Pioglitazone hydrochloride | 1.20 | 2.37 |
| equivalent to pioglitazone | | |
| Lactose monohydrate | 2.18 | 2.15 |
| Hydroxypropyl cellulose | 2.18 | 2.15 |
| Purified water | q.s. | q.s. |

Film Coating

| | | |
|---|---|---|
| Opadry ® White | 1.96 | 1.96 |
| Purified water | q.s. | q.s. |

Procedure:

### Core Tablets:

1. Metformin hydrochloride and sodium lauryl sulphate were mixed uniformly and granulated with a polyvinylpyrrolidone solution in purified water.
2. The granules were dried in a fluidized bed dryer and lubricated with magnesium stearate.
3. The blend of step 2 was compressed into core tablets.

### Seal Coating:

4. Hydroxypropyl methylcellulose was dispersed in isopropyl alcohol followed by the addition of purified water.
5. The tablets of step 3 were coated with the dispersion of step 4.

### ER Coating:

6. Cellulose acetate was dissolved in acetone followed by the addition of triacetin and polyethylene glycol under stirring.
7, Purified water was added slowly under stirring to the dispersion of step 6.
8. The seal coated tablets of step 5 were coated with the dispersion of step 7.

### Pioglitazone Layer:

9. Lactose and hydroxypropyl cellulose were dissolved in purified water.
10. Pioglitazone hydrochloride was dispersed into the solution of step 9.
11. The ER coated tablets were coated with the dispersion of step 10.

### Film Coating:

12. A film coating solution was prepared by dispersing Opadry® white in purified water.
13. The pioglitazone coated tablets were coated with the solution of step 12.

### Example 9

| | |
|---|---|
| Ingredients | Percent w/w of Tablet |

Core Composition

| | |
|---|---|
| Metformin hydrochloride | 70.25 |
| Polyvinylpyrrolidone | 5.78 |
| Sodium lauryl sulphate | 0.78 |
| Hydroxypropyl methylcellulose | 0.78 |
| Magnesium stearate | 0.78 |
| Purified water | q.s. |

Seal Coating (1.8% weight buildup)

| | |
|---|---|
| Hydroxypropyl methylcellulose | 1.41 |
| Isopropyl alcohol (85parts) | q.s. |
| Purified water (15 parts) | q.s. |

ER Coating (~10% weight build-up)

| | |
|---|---|
| Ethyl cellulose | 3.36 |
| Hydroxypropyl methylcellulose | 3.36 |
| Triethyl citrate | 1.01 |
| Isopropyl alcohol | q.s. |
| Purified water | q.s. |

Drug Coating

| | |
|---|---|
| Linagliptin | 0.39 |
| Metformin hydrochloride | 7.81 |
| Hydroxypropyl cellulose | 4.14 |
| Tween-80 | 0.16 |
| Purified water | q.s. |
| Ethanol | q.s. |

Procedure:

### Core Tablets:

1. Metformin hydrochloride, hydroxypropyl methylcellulose, and polyvinylpyrrolidone were mixed uniformly and granulated with purified water.
2. The granules were dried in a fluidized bed dryer and were mixed with hydroxypropyl methylcellulose and sodium lauryl sulphate followed by lubrication with magnesium stearate.
3. The blend of step 2 was compressed into core tablets.

### Seal Coating:

4. The tablets of step 3 were coated with a hydro-alcoholic solution of hydroxypropyl methylcellulose.

### ER Coating:

5. Ethyl cellulose, hydroxypropyl methylcellulose, and tri-ethyl citrate were dispersed in isopropyl alcohol:water.
6. The seal coated tablets of step 4 were coated with the dispersion of step 5.

### Twin Drug Layering:

7. Metformin hydrochloride and hydroxypropylcellulose were dissolved in purified water followed by the addition of Tween-80.
8. Linagliptin was dissolved in ethanol followed by the addition of the resulting solution to the solution of step 7.
9. The ER coated tablets were coated with the dispersion of step 8.

While several particular forms of the invention have been illustrated and described, it will be apparent that various modifications and combinations of the invention detailed in the text can be made without departing from the spirit and scope of the invention.

We claim:

1. A pharmaceutical composition of a biguanide and a low dose antidiabetic agent comprising:
(i) a biguanide core comprising a therapeutically effective amount of a biguanide or its pharmaceutically effective salts and one or more pharmaceutically acceptable excipients;
(ii) optionally a seal coat;
(iii) an extended-release coat comprising one or more rate-controlling materials wherein there is no passageway in the coat;
(iv) optionally a second seal coat;
(v) a low dose antidiabetic agent coat comprising a therapeutically effective amount of a low dose antidiabetic agent or its pharmaceutically effective salts and one or more pharmaceutically acceptable excipients; and
(vi) optionally a film coat.

2. The pharmaceutical composition according to claim 1, wherein the biguanide comprises metformin, phenformin, buformin, and pharmaceutically acceptable salts, solvates, polymorphs, enantiomers, isomers, or mixtures thereof.

3. The pharmaceutical composition according to claim 1, wherein the low dose antidiabetic agent is selected from DPP-IV inhibitors, meglitinides, second generation sulphonylureas, glucagon-like peptide (GLP-1) analogues, other hypoglycemics which are used as an adjunct to metformin therapy, or mixtures thereof.

4. The pharmaceutical composition according to claim 1, wherein the biguanide is metformin and the low dose antidiabetic agent is a DPP-IV inhibitor.

5. The pharmaceutical composition according to claim 1, wherein the biguanide is layered onto a pharmaceutically inert core or seed.

6. The pharmaceutical composition according to claim 5, wherein the inert core or seed is hydrosoluble or hydroinsoluble.

7. The pharmaceutical composition according to claim 1, wherein the biguanide core comprises one or more pharmaceutically acceptable excipients selected from one or more of fillers, binders, disintegrants, anti adherents, lubricants, glidants, osmogents, coloring agents, and flavoring agents.

8. The pharmaceutical composition according to claim 1, wherein the biguanide core additionally contains one or more absorption enhancers.

9. The pharmaceutical composition according to claim 1, wherein the biguanide core additionally contains one or more swellable polymers.

10. The pharmaceutical composition according to claim 1, wherein the seal coat is applied over the biguanide core or over the extended-release coat.

11. The pharmaceutical composition according to claim 1, wherein the biguanide core further comprises a low dose antidiabetic agent.

12. The pharmaceutical composition according to claim 1, wherein the low dose antidiabetic agent coat further comprises a biguanide.

13. The pharmaceutical composition of claim 1, wherein the biguanide core further comprises a low-dose antidiabetic agent, and the low-dose antidiabetic agent coat further comprises a biguanide.

14. The pharmaceutical composition according to claim 1, wherein the rate-controlling material is selected from one or more of hydrophilic polymers, hydrophobic polymers, water swellable polymers, other hydrophobic materials, or mixtures thereof.

15. A pharmaceutical composition of a biguanide and a low dose antidiabetic agent that includes:
(i) a biguanide core which includes a therapeutically effective amount of biguanide or its pharmaceutically effective salts and one or more pharmaceutically acceptable excipients;
(ii) optionally a seal coat;
(iii) an extended-release coat which includes one or more rate-controlling materials wherein there is no passageway in the coat;
(iv) optionally a second seal coat;
(v) an immediate-release drug coat which includes a biguanide and a low dose antidiabetic agent or its pharmaceutically effective salt and one or more pharmaceutically acceptable excipients; and
(vi) optionally a film coat.

16. The pharmaceutical composition according to claim 15, wherein the ratio of biguanide in the core to that in the coat ranges from about 99:1 to about 60:40.

17. A process for preparing a pharmaceutical composition comprising the steps of:
(i) preparing the cores which include a biguanide or its pharmaceutically effective salts and one or more pharmaceutically acceptable excipients;
(ii) optionally coating the cores with a seal coat;
(iii) coating the biguanide cores of step (ii) with an extended-release coating composition which includes one or more rate-controlling materials;
(iv) coating the extended-release core with an immediate-release coating composition which includes a low dose antidiabetic agent or its pharmaceutically effective salts and one or more pharmaceutically acceptable excipients; and
(v) optionally providing a film coat.

18. A method for treating diabetes by administering to a person in need thereof a pharmaceutical composition of a biguanide and a low dose antidiabetic agent comprising:
(i) a biguanide core which includes a therapeutically effective amount of biguanide or its pharmaceutically effective salts and one or more pharmaceutically acceptable excipients;
(ii) optionally a seal coat;
(iii) an extended-release coat which includes one or more rate-controlling materials wherein there is no passageway in the coat;
(iv) a low dose antidiabetic agent coat which includes a therapeutically effective amount of a low dose antidiabetic agent or its pharmaceutically effective salts and one or more pharmaceutically acceptable excipients; and
(v) optionally a film coat.

19. The method for treating diabetes according to claim 18, wherein the low dose antidiabetic agent is a DPP-IV inhibitor.
